SEQUENCE LISTING (2 sequences)

SEQ ID NO: 1 - DNA, 1440 bp, Escherichia coli
  CDS (100)..(1437)
tcgactgcataaggagccgggcgtttaagcaccccacaaaacacacaaagcctgtcacaggtgatgtgaaaaaagaaaagcaatgactcaggagatagaatgatgattactctgcgcaaacttcctctggcggttgccgtcgcagcgggcgtaatgtctgctcaggcaatggctgttgatttccacggctatgcacgttccggtattggttggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcacaacagaatgactgggaagctaccgatccggccttccgtgaagcaaacgtgcagggtaaaaacctgatcgaatggctgccaggctccaccatctgggcaggtaagcgcttctaccaacgtcatgacgttcatatgatcgacttctactactgggatatttctggtcctggtgccggtctggaaaacatcgatgttggcttcggtaaactctctctggcagcaacccgctcctctgaagctggtggttcttcctctttcgccagcaacaatatttatgactataccaacgaaaccgcgaacgacgttttcgatgtgcgtttagcgcagatggaaatcaacccgggcggcacattagaactgggtgtcgactacggtcgtgccaacttgcgtgataactatcgtctggttgatggcgcatcgaaagacggctggttattcactgctgaacatactcagagtgtcctgaagggctttaacaagtttgttgttcagtacgctactgactcgatgacctcgcagggtaaagggctgtcgcagggttctggcgttgcatttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgtcggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtcttcgcaacctacgccaagtgggatgagaaatggggttacgactacaccggtaacgctgataacaacgcgaacttcggcaaagccgttcctgctgatttcaacggcggcagcttcggtcgtggcgacagcgacgagtggaccttcggtgcccagatggaaatctggtggtaa

SEQ ID NO: 2 - protein, 446 aa, Escherichia coli [sequence omitted]

(19) United States
(12) Patent Application Publication
(10) Pub. No.: US 2003/0096223 A1
(43) Pub. Date: May 22, 2003

(54) Screening system to identify polynucleotides encoding cleavable N-terminal signal sequences

(76) Inventors: Alan L. Greener, San Diego, CA (US); Hwai Wen Chang, San Marcos, CA (US); Carsten Peter Carstens, San Diego, CA (US)

Correspondence Address: FINNEGAN, HENDERSON, FARABOW, GARRETT &, DUNNER LLP, 1300 I STREET, NW, WASHINGTON, DC, 20006, US

(21) Appl. No.: 09/794,213

(22) Filed: Feb. 28, 2001

Related U.S. Application Data
(60) Provisional application No. 60/185,560, filed on Feb. 28, 2000.

(51) Int. Cl. C12Q 1/70; G01N 33/53; G01N 33/554; G01N 33/569; C12N 15/86

(52) U.S. Cl. 435/5; 435/7.1; 435/7.32; 435/456

(57) ABSTRACT

The present invention relates to methods for the identification of polynucleotides that encode cleavable N-terminal signal sequences.

### RELATED APPLICATION INFORMATION

This application claims the filing date benefit of U.S. Provisional Patent Application Ser. No. 60/185,560, filed Feb. 28, 2000, which is incorporated by reference herein in its entirety for any purpose.

### 1.0 FIELD OF THE INVENTION

The present invention relates to methods to identify proteins with cleavable N- terminal signal sequences and polynucleotides encoding those proteins.

### 2.0 BACKGROUND AND SUMMARY OF THE INVENTION

Intracellular and intercellular communication is central to the proper functioning of an organism. Such communication often may occur via proteins that belong to a variety of classes, for example, hormones, cytotoxic factors or growth factors. Also, cellular communication often relies on membrane receptors to transmit signals (e.g., signal transduction). These classes of proteins are usually synthesized containing an N-terminal leader peptide (i.e., signal peptide) that may be specifically cleaved off as the proteins are directed to their membrane or an extracellular location.

In order to better understand signal transduction pathways and other cellular events that involve secreted or membrane proteins, it would be important to identify and characterize as many as possible of the signal transduction proteins found in a cell or organism. Screening techniques developed in the biotechnological sciences provide tools for the identification and characterization of proteins and their corresponding polynucleotides.

Genetic selection or screening systems to discover signal transduction proteins have been described. Such systems have employed yeast or mammalian cells as host organisms (i.e., the organism in which polynucleotide clones are screened). (Yeast (Jacobs et al., 1999, Meth. Enzymol. 303:468-479, Jacobs et al., 1997, Gene 198:289-296; Klein et al., 1996, Proc. Natl. Acad. Sci. USA 93:7108-7113; Singh Sidhu et al., 1991, Gene 107:111-118), COS cells (Arca et al., 1999, Proc. Natl. Acad. Sci. USA 96:1516-1521; Sugano et al., 1998, DNA Res. 5:187-193; Kristofferson et al., 1996, Anal. Biochem. 243:127-132; Shirozu et al., 1996, Genomics 37:273-280; Yokoyama-Kobayashi et al., 1995, Gene 163:193-196; Tashiro et al., 1993, Science 261:600-602), murine B cells (Kojima et al., 1999, Nature Biotechn. 17:487-490), mouse stromal cells (Hamada et al., 1996, Gene 176:211-214) or murine hemopoietic cells (Zannettino et al., 1996, J. Immunol. 156:611-620)) Also, Drosophila embryos have been used in hybridization based screening (Kopczynski et al., 1998, Proc. Natl. Acad. Sci. USA 95:9973-9978). Although these systems are useful in selectively isolating genes encoding membrane receptors and secreted proteins, they have important drawbacks. For example, cDNA libraries constructed for screening in these eukaryotic organisms must undergo a number of generations of amplification in E. coli, which potentially introduces a significant bias in the selection of random clones. In addition, the ability to introduce high complexity libraries into yeast or mammalian cells is significantly less efficient than performing the selection directly in E. coli. Finally, these eukaryotic systems were not designed to study secreted proteins and membrane receptors of prokaryotic organisms that have been shown or are suspected to be directly or indirectly involved in human, animal and/or plant pathogenicity.

An E. coli selection system that does not require amplification to identify polynucleotides encoding signal transduction proteins would allow the screening of significantly more complex libraries with reduced clonal bias. In addition, a bacterial system has the advantage that genomic libraries of prokaryotic organisms can be directly screened for signal transduction proteins. This selection may be important in identifying therapeutic targets present on the surface of bacteria, particularly those that are pathogenic to mammals and plants.

Attempts to develop an E. coli system to identify signal transduction proteins have been reported (Giladi et al., 1993, J. Bacteriol. 175:4129-4136; Blanco et al., 1991, Mol. Microbiol. 5:2405-2415; Boquet et al., 1987, J. Bacteriol. 169:1663-1669). These systems used periplasmically localized reporter genes (βlactamase or alkaline phosphatase) (For example, a phosphatase marker (alkaline phosphatase: Giladi et al., 1993, supra; Blanco et al., 1991, supra; acid phosphatase: Boquet et al., 1987, supra)). Those systems, however, identified transmembrane domains that did not include cleavable N-terminal regions. The transmembrane domains would permit the periplasmic localization of the vector proteins (allowing them to be active) even though they remained tethered to the inner membrane by the transmembrane fusion segments.

It would be advantageous to have a screening system designed to identify proteins with cleavable N-terminal signal peptides. The present invention provides such a screening system.

The present invention relates to methods to identify proteins with a cleavable N-terminal signal sequence and polynucleotides encoding those proteins. Using the methods of the invention, one may screen large numbers of polynucleotide clones to identify a clone which corresponds to a polynucleotide that encodes a protein with a cleavable N-terminal signal sequence.

In certain preferred embodiments, the methods of the invention use prokaryotic cells to identify new polynucleotides. Polynucleotides are screened using the described methods by expressing the polynucleotides in prokaryotic cells.

In preferred embodiments, the polynucleotides are expressed in prokaryotic cells together with a selectable marker that facilitates the identification of polynucleotides encoding a cleavable N-terminal signal sequence. Such markers preferably are cell surface proteins that can be used to distinguish between cells that have such surface proteins and cells that do not have such surface proteins. Such surface proteins may confer a property on cells, such as a cell surface receptor property that facilitates interactions or uptake of other molecules, phages, or viruses. Such a property may include conferring on a cell the ability to be infected by a particular phage or virus.

It may also confer on the cell the ability to uptake a particular nutrient. For example, those skilled in the art are aware of cell surface receptors that are required for a cell to take in a particular sugar. If cells are grown in a media that includes only that sugar as a carbon source, one can determine whether the cells have the required cell surface receptor. Similarly, those skilled in the art are aware of other receptors for other nutrients such as vitamins.

The cell surface protein may also be detected by an interaction with another molecule. For example, one may detect the presence or absence of a particular cell surface protein by the cells ability to bind to a ligand, such as an antibody specific for the cell surface protein.

In certain embodiments, the cells used to screen polynucleotides are designed such that they do not have the cell surface protein unless a polynucleotide encoding a cleavable N-terminal signal sequence is fused to a polynucleotide encoding the cell surface protein. Such cells may include polynucleotides encoding the cell surface protein that lack sequence encoding a cleavable N-terminal signal sequence. One can determine the presence of a nucleotide encoding a cleavable N-terminal signal sequence in such embodiments by fusing a polynucleotide being screened to the polynucleotide encoding the cell surface protein. One will detect the presence of a screened polynucleotide encoding an N-terminal signal sequence if the cell surface protein is detected on the cells. As discussed above, the presence of the cell surface protein may be detected by a change in a functional property of the cell, such as the ability to be infected by a phage or a virus or the ability to take in a nutrient. Such presence may also be detected by interaction of the cell surface protein with another molecule such as an antibody or other ligand.

In certain preferred embodiments, the selectable marker used in the described methods is the bacteriaphage lambda receptor (lamB) or an analog thereof. Cells are used that include polynucleotides encoding lamB or an analog that lacks sequence encoding an N-terminal signal sequence is fused to polynucleotide to be screened. If the polynucleotide being screened encodes an N-terminal signal sequence, the lamB protein or analog will be included as a receptor on the cell surface, which will result in cells that can be infected with a phage or virus. If the polynucleotide being screened does not encode an N-terminal signal sequence, the lamB protein or analog will not be included as a receptor on the cell surface, which will result in cells that cannot be infected with a phage or virus.

The prokaryotic cell expressing the fusion protein is exposed to a phage or virus. Preferably, the phage or virus carries a selectable marker, for example an antibiotic resistance marker, which confers a selectable property to the prokaryotic cell if it is infected with the phage or virus. In preferred embodiments, a polynucleotide encoding a cleavable N-terminal signal sequence is identified by selecting for prokaryotic cells that have been infected with the phage or virus that carries a selectable marker.

### 3.0 BRIEF DESCRIPTION OF THE FIGURES

FIG. 1 shows the polynucleotide and amino acid sequence of lamB (SEQ ID NOS:1 and 2) (Genbank Accession Nos. M26131, M26187). The signal sequence is underlined.

FIG. 2 shows the map of the pKK LamB-E vector.

FIG. 3 shows the map of the pKK LamB-P vector. There are three versions of pKK LamB-P (1, 2, and 3) to accomodate all three reading frames. FIG. 3 shows version 1. Version 2 includes one base added after the Xbal site and before the LamB portion. Version 3 includes two bases added after the Xbal site and before the LamB portion.

### 4.0 DETAILED DESCRIPTION

The present invention relates to a method that facilitates the identification of proteins with a cleavable N-terminal signal sequence. The term “protein” in this application refers to a segment of covalently linked amino acids of at least 2 amino acids in length. Thus, the term “protein” is used to refer to a protein, a polypeptide and a peptide, which may be modified or in its native form, unless the context indicates otherwise.

The term “signal sequence” refers to a stretch of amino acids that is capable of effecting the localization of a protein in the periplasmatic space, the cell membrane, the outer cell membrane, the extracellular space or more than one of these locations inside or outside the cell. A signal sequence typically is part of the N-terminal portion of a protein. A signal sequence typically is from about 5 to about 50 amino acids in length, and in certain embodiments, typically is about 20 amino acids.

The term “native form” refers to the form of a protein that results from the translation of the open reading frame of the messenger RNA (“mRNA”) that encodes the protein.

The word “cleavable”, when used in connection with a signal sequence, means that the signal sequence can be cleaved off when fused to a marker protein that is used in the methods described herein, unless the context indicates otherwise. However, a cleavable signal sequence may or may not be cleaved off a protein when that protein is not fused to a marker protein used in the methods described herein.

For example, pathogenic bacteria (or plasmids contained within pathogenic bacertia) may encode proteins called invasins, which directly elicit a cytotoxic response. See, e.g., Cornelius, G.R., 1998, J. Bacteriol. 180:5495-5504. Invasins that are secreted into a mammalian target cell are directed to their extracellular location by a mechanism that does not utilize traditional N-terminal signal peptide (sec-dependent) that is naturally cleaved. See, e.g., Hueck, C. J., 1998, Micro. Mol. Biol. Rev. 6:379-433. This secretion is known as Type III secretion. See, e.g., Hueck, C. J., 1998, Micro. Mol. Biol. Rev. 6:379-433. The invasins, however, contain a particular motif at their N-terminus that directs them for secretion even though there is no natural cleavage. Fusions between the N-terminus of an invasin and another protein, however, can result in secretion of the fusion protein with cleavage of the motif at the N-terminus that is not naturally cleaved without such a fusion. See, e.g., Michiels et al., 1991, J. Bacteriol. 173:1677-1685.

The methods of the present invention can identify proteins with a cleavable N-terminal signal sequence in their native form and polynucleotides encoding such proteins. A signal sequence is found in a variety of protein families. Typical proteins with a cleavable N-terminal signal sequence are found in a peripheral cellular location (e.g., the cell membrane, the periplasmatic space, the outer cell membrane). Such proteins may be found in the extracellular space, after the protein has completed the processes of translation and posttranslational processing. Examples of proteins which can be identified using the described methods include, but are not limited to, eukaryotic proteins (e.g., hormones, growth factors, membrane receptors, secreted proteins, cell surface receptors, transport proteins, etc.) and prokaryotic proteins (e.g., invasins, cell surface receptors, transport proteins, periplasmically localized enzymes, etc.). These proteins are involved in many critical cellular phenomena. Thus, the screening methods described herein provide a valuable tool to identify proteins that are useful for many purposes, including but not limited to, therapeutics and diagnostics.

The methods of the present invention facilitate the screening of many polynucleotide clones through the use of a selectable marker. According to certain embodiments, the selectable marker is a cell surface protein, which is not included on the cell surface of the cells employed in the process unless a polynucleotide encoding a cleavable N-terminal signal is present. The polynucleotide being screened is fused to polynucleotide encoding the cell surface receptor. If the polynucleotide being screened does not include sequence encoding a cleavable N-terminal signal sequence, the selectable marker is not secreted and is not included on the cell surface. If the screened polynucleotide encodes a cleavable N-terminal signal sequence, it would be cleaved off when the fusion protein is expressed in a suitable prokaryotic cell. The processing of the fusion protein by a prokaryotic cell is then detected by determining whether the cell surface protein is included on the cell surface. That may be accomplished by testing for appropriate cell surface receptor activity or by detecting the cell surface protein by its binding to a ligand, such as an antibody.

In certain embodiments, a screening cassette is used in the methods of the invention. Such screening cassettes may include a selectable cell surface marker and a multiple cloning site for insertion of a screened polynucleotide sequence. When introduced into a prokaryotic cell, the screening cassette would direct the expression of the selectable cell surface marker protein and the protein encoded by the screened polynucleotide in the form of a fusion protein. In preferred embodiments, the cell surface marker protein is located C-terminal to the screened protein in the fusion protein. If all or part of the screened polynucleotide encodes a cleavable N-terminal signal sequence, the cell surface marker protein would be present on the cell surface, where its presence can be detected.

4(A) Screening Cassettes

The methods of the present invention facilitate the screening of large numbers of polynucleotides to identify proteins with a cleavable N-terminal signal sequence. In certain embodiments, a screening cassette is used for the screening of polynucleotides. Screening cassettes useful for the methods of the invention preferably comprise an open reading frame (“ORF”) that includes a polynucleotide encoding a selectable cell surface marker. In certain embodiments, the ORF further comprises a multiple cloning site for insertion of a polynucleotide that is screened. In preferred embodiments, the multiple cloning site is located upstream of the cell surface marker polynucleotide. In certain preferred embodiments, the multiple cloning site allows the insertion of the screened polynucleotide following digestion with different restriction endonucleases, so that the screened polynucleotide and the cell surface marker polynucleotide are in frame for at least one of these insertions. In certain preferred embodiments, three screening cassettes are used in which the cell surface marker polynucleotide is found in each of the three reading frames.

In certain embodiments, the screening cassette comprises elements that facilitate the expression of the polynucleotides of the ORF (for example, expression of the cell surface marker polynucleotide and the screened polynucleotide) in a prokaryotic cell. In certain embodiments, the screening cassette comprises elements that facilitate selection for the presence of the cassette in a prokaryotic cell. In certain embodiments, the screening cassette may be part of a vector which may comprise elements to facilitate the propagation of the vector in a prokaryotic cell. In further embodiments, the screening cassette may or may not integrate into the genomic DNA of the host prokaryotic cell.

4(A)(1) Selectable Markers Useful for the Screening Cassettes

In certain embodiments, the methods of the present invention use a selectable cell surface marker that can be detected on the cell surface if the polynucleotide being screened encodes a cleavable signal sequence. Most preferably, the fusion protein comprises the selectable cell surface marker and a protein that is encoded by a polynucleotide which is screened using the methods of the invention. For example, in certain preferred embodiments, the fusion protein comprises a cleavable N-terminal protein sequence encoded by the polynucleotide that is screened and a C-terminal protein sequence encoded by the marker polynucleotide.

In certain preferred embodiments, the fusion protein comprising the selectable marker is expressed in a prokaryotic cell. In certain embodiments, following translation of the ORF comprising the cell surface marker polynucleotide, a fusion protein comprising the marker protein and a screened N-terminal sequence is expressed in the prokaryotic cell. The fusion protein can be processed by a mechanism for posttranslational protein modifications of the prokaryotic cell provided the fusion protein contains the necessary characteristics. For example, if the N-terminal sequence that is screened has, at least in part, the characteristics of a cleavable N-terminal signal sequence, all or a part of the fusion protein that is encoded by the screened polynucleotide is cleaved off.

IIf the cell surface marker protein is expressed as part of a fusion protein that is C- terminal to the protein encoded by the screened polynucleotide, the screened protein sequence will be identified as encoding, at least in part, a cleavable N-terminal signal sequence through detection of the cell surface marker protein on the cell surface.

4(A)(2) LamB

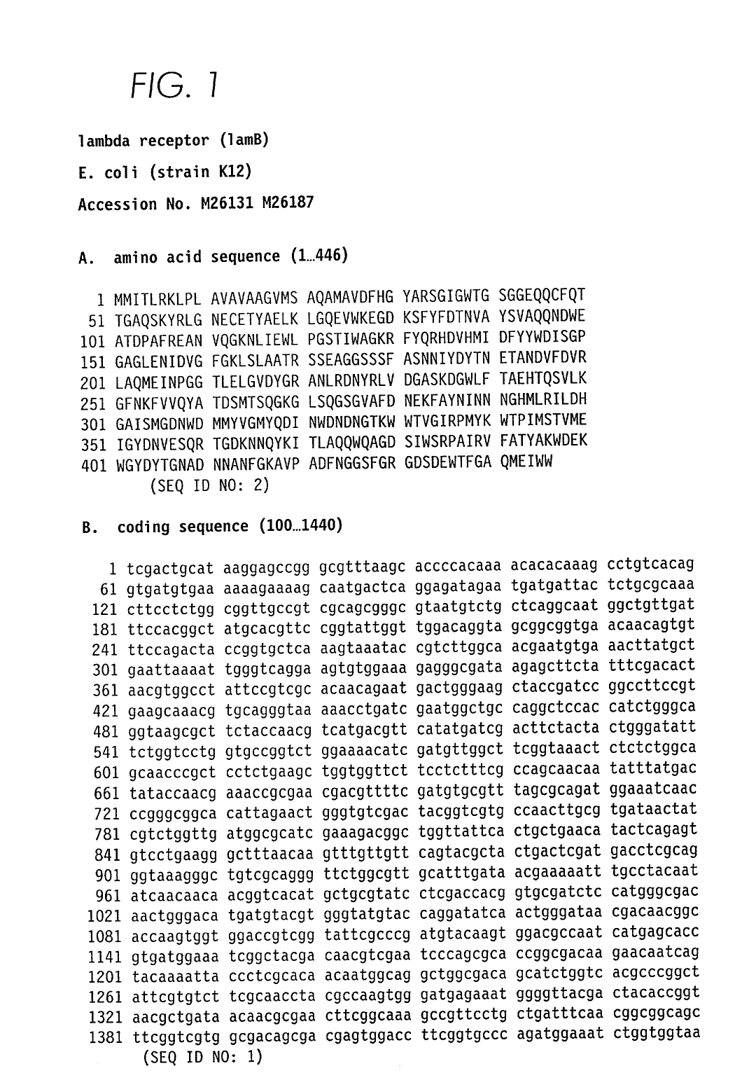

In certain preferred embodiments, the lamB protein is used as a selectable marker in the described methods. The term “lamb protein” as used herein means a protein as shown in FIG. 1 or homologues or derivatives thereof as discussed herein, unless the context indicates otherwise. In certain preferred embodiments, the lamB protein used in the present invention has a protein sequence and a corresponding polynucleotide sequence as shown in FIG. 1 (SEQ ID NOS:1 and 2)(Genbank Accession Nos. M26131, M26187). Every polynucleotide that encodes the lamB protein shown in FIG. 1 can also be used as a selectable marker in the described methods.

The lamB gene encodes a protein that can function as a receptor for bacteriophage lambda. When that protein is present on the cell surface of E. coli, the host is sensitive to lambda infection. When that protein is absent or mutated in certain ways, the E. coli are resistant to lambda infection. If the lamB gene is changed so that it does not encode an N-terminal signal sequence, the protein will not be translocated to the periplasm and, thus, will not be inserted into the outer membrane. E. coli cells having only such lamB genes without sequences encoding a cleavable N-terminal signal sequence have no to little ability of being infected by lambda phage.

According to certain embodiments of the invention, screening cassettes are employed that comprise lamB genes without sequences encoding an N-terminal signal sequence. Thus, during the screening, if the screening cassette is fused to another gene that encodes a cleavable N- terminal signal sequence, the fusion protein will translocate to the periplasm and be inserted into the outer membrane. Such cells will then become sensitive to lambda phage infection. If there is no fusion to another gene encoding a cleavable N-terminal signal sequence, the protein encoded by the lamB gene will not be inserted into the outer membrane, and the cell will have no or little infection by lambda phage.

In certain preferred embodiments, a polynucleotide sequence that is screened is ligated to a polynucleotide encoding the lamB protein without a cleavable N-terminal signal sequence. The resulting polynucleotide encodes a fusion protein. When that fusion protein is expressed in a prokaryotic cell (e.g., E. coli), the protein sequence encoded by the screened polynucleotide, or at least the part of it which corresponds to a cleavable N-terminal signal sequence, would be cleaved off the fusion protein. The resulting protein would be the lamB protein and possibly some amino acid residues encoded by the screened polynucleotide which were not cleaved off. Therefore, the fusion protein would be processed in the prokaryotic cell to remove a cleavable N-terminal signal sequence, which results in enhanced phage receptor activity of the lamB protein. In other words, cells that previously would have no to little infection by lambda phage, become sensitive to such infection as a result of the screened polynucleotide, which encodes a cleavable N-terminal signal sequence.

In certain preferred embodiments, the screened polynucleotide sequence that is ligated to a polynucleotide encoding the lamB gene is not larger in size than about 1,500 base pairs, more preferably not more than about 800 base pairs, more preferably about 600 base pairs, more preferably about 400 base pairs and most preferably about 200 base pairs. The mininum size of the screened polynucleotide sequence is at least about 50 base pairs, more preferably at least about 100 base pairs and more preferably at least about 150 base pairs.

When using the lamB protein as a marker in the described methods, one screens for polynucleotides encoding a cleavable N-terminal signal sequence by exposing the prokaryotic cells used in the screen with a phage or virus. After expressing a fusion protein comprising a screened protein sequence and the lamB protein in a prokaryotic cell, one can detect the presence of a screened protein sequence containing a cleavable N-terminal signal sequence when the cells show increased infection by a phage that recognizes the lamB protein compared to cells that do not include fused screened proteins.

Thus, if the screened polynucleotide encodes, at least in part, a cleavable N-terminal signal sequence, the fusion protein will be processed when expressed in a prokaryotic cell. Once processed, the mature protein will contain the lamB protein. In addition, the mature protein may contain additional amino acid residues that were encoded by the screened polynucleotide but not cleaved off during posttranslational processing (e.g., amino acid residues that are not part of a signal sequence encoded by the screened polynucleotide). The phage or virus will have a higher rate of infection of cells that include a cleavable N-terminal signal sequence fused to lamB protein than to cells that include lamB protein that is not fused to such a cleavable signal sequence.

In certain embodiments, when polynucleotides are expressed as lamB fusions in a prokaryotic cell, the cell is infected with a phage or virus that confers a detectable property to the cell. Thus, in certain embodiments, one employs cells that lack a selectable property that can be conferred through phage or viral infection. Examples of such selectable properties are resistance to an antibiotic, for example, chloramphenicol, streptomycin, ampicillin, erythromycin, kanamycin (neomycin), tetracycline gentamycin, and hygromycin (Davies et al, 1978, Annu. Rev.Microbiol. 32:469), etc. In certain embodiments, a biosynthetic gene, such as those in the histidine, tryptophan, and leucine biosynthetic pathways may be conferred through infection by the phage or virus. In other embodiments, the screening is carried out by conferring an activity that can produce or process a dye, such as β-galactosidase, alkaline phosphotase or a fluorescent protein.

Thus, in preferred embodiments, lamB expression in the prokaryotic cells used in the described methods is identified through infection with a phage or virus which confers a detectable property to the cells which they otherwise lack.

In certain embodiments, the processing of the lamB fusion protein may be screened by detecting the presence of the mature lamB protein in the outer cell membrane of a prokaryotic cell expressing the lamB fusion protein. LamB is a protein found in the outer cell membrane of prokaryotes. Schulein et al., 1990, Mol. Microbiol. 4:625-632; Element et al., 1981, Cell 27:507-514. A signal sequence is required for the lamB protein to be located in the cell membrane. Altman et al., 1990, J. Biol. Chem. 265:18148-18153; Altman et al., 1990, J. Biol. Chem. 265:18154-18160. Furthermore, processing of the signal sequence is required for the mature lamB protein to be located in the outer cell membrane of a prokaryotic cell. Carlson et al., 1993, J. Bacteriol. 175:3327-3334.

In such embodiments, one employs an expression cassette that encodes lamB without a. signal sequence as discussed above. Thus, unless the screened polynucleotide encodes a cleavable N-terminal signal sequence that is fused to the lamB, the lamB will not be exported to the outer cell membrane. The presence of a polynucleotide encoding a cleavable N-terminal signal sequence, can therefore be determined by detecting lamb protein in the outer cell membrane. For such detection, one can employ any type of antibody against the lamB protein. Antibodies that can be used include, but are not limited to, polyclonal antibodies, monoclonal antibodies, humanized antibodies, chimeric antibodies, single-chain antibodies, FAB fragments, etc. See, e.g., Antibodies: A Laboratory Manual, ed. by Harlow and Lane (Cold Spring Harbor Press: 1988) and references therein, which discuss the preparation of antibodies. Preferably, the antibody is specific to a domain of the lamB protein that is easily accessible, for example, the extracellular domain (Molla et al., 1989, Biochemistry 28:8234-8241; Schenkman et al., 1984, J. Biol. Chem. 259:7570-7576). In another embodiment, an epitope tag is attached to the lamB protein, preferably to the extracellular domain, which can be easily identified using an antibody. An example of such an epitope tag is the FLAG epitope tag (Hopp et al., 1988, Biotechnology 6:1204-1210).

4(A)(3) LamB Analogs

In other embodiments, an analog of the lamB protein is used as a selectable marker in the described methods. As used herein, the term “analog of the lamB protein” refers to a protein that is capable of facilitating infection of a prokaryotic cell by a phage or virus.

One may test whether a lamB analog is capable of facilitating the infection of a prokaryotic cell by a phage or virus. For example, one may express a lamB analog in E. coli cells that are deficient in the analog, i.e., cells that cannot be infected with the phage or virus prior to expression of the analog. These E. coli cells that express the lamB analogs are then contacted with a phage or virus strain that carries a selectable marker that is not expressed in the E. coli cells prior to infection by the phage or virus. If the cells are infected, they aquire a new resistance marker and can therefore be readily identified. This strategy can be readily employed by the skilled artisan to identify lamB analogs, or to test known lamB analogs for their functional utility for the described methods. Also, this strategy may be used for any strain, species, family, genus, order, class or phylum of prokaryotic cells. Methods that can be used for for analyzing lamB analogs are also discussed in Hofnung et al., 1981, J. Bacteriology 148:853-860 and Element et al., 1982, Ann. Microbiol. 133A:9-20. Another example is the fhuA gene product which serves as the receptor for the bacteriophages T1 and φ80 (Coulton et al., J. Bacteriol., 156:1315-1321 (1981)).

In addition to having the functional similarity to lamB protein by rendering cells cabable of being infected by phages or viruses, lamB protein analogs may also be structural homologues of the lamB protein. Such homologs may include conservative changes from the lamB protein. Conservative changes include, for example, substitutions, additions and/or deletions of amino acid residues that do not render the protein incapable of facilitating the infection of a prokaryotic cell by a phage or virus in the methods of the present invention. For example, substituting, adding, and/or deleting one or more amino acid residues of the lamb protein may result in a silent change. As used herein, the term “silent change” refers to a change in amino acid sequence of a protein that does not render the protein useless for the described methods.

A silent change can be made, for example, by substituting an amino acid residue with another residue with similar charge, polarity, solubility, hydrophobicity, hydrophilicity, or a similar amphipathic nature. For example, amino acids with uncharged polar head-groups that have similar hydrophilicity values include glycine, asparagine, glutamine, serine, threonine and tyrosine; and amino acids with nonpolar head-groups include alanine, valine, isoleucine, leucine, phenylalanine, proline, methionine, tryptophan; negatively charged amino acids include aspartic acid and glutamic acid; positively charged amino acids include lysine, histidine and arginine.

Whether a change in the sequence shown in FIG. 1 is conservative or not can also be evaluated by the skilled artisan using analytical tools known in the art. For example, algorithms useful to predict protein structures (e.g., secondary and/or tertiary structures) can be employed to predict the effect of a sequence change, for example, the Chou-Fasman method. Also helpful, for example, is an analysis using a Ramachandran plot to predict the effect of a sequence change on the structure of the protein.

When evaluating lamB homologues or when designing lamB homologues, a skilled artisan would be guided by what is known about the wild-type lamB protein. For example, the three dimensional structure of the lamB protein has been determined (Schirmer et al., 1995, Science 267:512-514). Thus, the location of a particular amino acid residue in the overall structure of the lamB protein can be used to evaluate how critical it is to functions related to phage or viral infection.

Also useful in analyzing lamB homologues are Werts et al., 1994, J. Bacteriol. 176:941-947; Charbit et al., 1994, J. Bacteriol. 176:3204-3209; Ferenci et al., 1989, FEMS Micro. Lett. 61:335-340; Charbit et al., 1988, J. Mol. Biol. 201:487-496; Gehring et al., 1987, J. Bacteriol. 169:2103-2106 and Charbit et al., 1984, J. Mol. Biol. 175:395-401, which discuss amino acid residues in the lamb protein that are important to facilitate phage infection. Also helpful are Chan et al., 1996, Mol. Membrane Biol. 13:41-48; Carlson et al., 1993, J. Bacteriol. 175:3327-3334; Altman et al., 1990, J. Biol. Chem. 265:18148-18153; Altman et al., 1990, J. Biol. Chem. 265:18154-18160; Molla et al., 1989, Biochemistry 28:8234-8241; Heine et al., 1987, Gene 53:287-292; Boulain et al., 1986, Mol. Gen. Genet. 205:339-348 and Schenkman et al., 1984, J. Biol. Chem. 259:7570-7576, which provide functional analysis of different regions of the lamB protein. Further references on lamB protein are Element et al., 1981, Cell 27:507-514, which discusses the sequence and domain structure of lamB; De Vries et al., 1984, Proc. Natl. Acad. Sci. USA 81:6080-6084, which discusses the isolation of constitutively expressed lamB genes; Schulein et al., 1990, Mol. Microbiol. 4:625-632, which discusses lamB protein from Salmonella typhimurium.

In some embodiments, a homologue of the lamB protein useful in the described methods is preferably at least about 70% identical to the sequence shown in FIG. 1, more preferably at least about 80%, more preferably at least about 85%, more preferably at least about 90%, more preferably at least about 95% and most preferably at least about 98 to 99%.

In further embodiments, a lamB homologue is encoded by a polynucleotide that is at least about 50% identical to a polynucleotide which encodes the protein shown in FIG. 1, more preferably at least about 65%, more preferably at least about 80%, more preferably at least about 90%, more preferably at least about 95% and most preferably at least about 98 to 99%.

Percent identity involves the relatedness between amino acid or nucleic acid sequences. One determines the percent of identical matches between two or more sequences with gap alignments that are addressed by a particular method. The percent identity may be determined by visual inspection and mathematical calculation. Alternatively, the percent identity of two nucleic acid sequences can be determined by comparing sequence information using the GAP computer program, version 6.0 described by Devereux et al. (Nucl. Acids Res. 12:387, 1984) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp.353-358, 1979; (2) apenalty of 3.0 for each gap and an additional 0.10 penalty for each symbol in each gap; and (3) no penalty for end gaps. Other programs used by one skilled in the art of sequence comparison may also be used.

In certain embodiments, lamb homologue nucleic acids may be those that hybridize under moderately or highly stringent conditions to the complement of naturally-occurring lamB encoding nucleic acids or to nucleic acids that encode lamB proteins having naturally-occurring amino acid sequences. As used herein, conditions of moderate stringency can be readily determined by those having ordinary skill in the art based on, for example, the length of the DNA. The basic conditions are set forth by Sambrook et al. Molecular Cloning: A Laboratory Manual, 2 ed. Vol. 1, pp. 1.101-104, Cold Spring Harbor Laboratory Press, (1989), and include use of a prewashing solution for the nitrocellulose filters 5× SSC, 0.5% SDS, 1.0 MM EDTA (pH 8.0), hybridization conditions of about 50% formamide, 6× SSC at about 42° C. (or other similar hybridization solution, such as Stark's solution, in about 50% formamide at about 42° C.), and washing conditions of about 60° C., 0.5× SSC, 0.1% SDS. Conditions of high stringency can also be readily determined by the skilled artisan based on, for example, the length of the DNA. Generally, such conditions are defined as hybridization conditions as above, and with washing at approximately 68° C., 0.2× SSC, 0.1% SDS. The skilled artisan will recognize that the temperature and wash solution salt concentration can be adjusted as necessary according to factors such as the length of the probe.

In yet further embodiments, a lamB homologue useful for the described methods is encoded by a polynucleotide that is capable of hybridizing to a second polynucleotide wherein the second polynucleotide is complementary to a polynucleotide which encodes the protein shown in FIG. 1. Hybridization conditions are well known in the art, see, for example, Sambrook et al., 1989, Molecular Cloning, A Laboratory Manual, Cold Springs Harbor Press, New York; and Ausubel et al., 1989, Current Protocols in Molecular Biology, Green Publishing Associates and Wiley Interscience, New York. For example, hybridization may be carried out in 6× SSC at about 45° C. Following the hybridization step, one may wash in about 4-5× SSC at 50° C. for low stringency hybridization, more preferably in about 2-3× SSC at 50° C. for moderate stringency hybridization and most preferably in about 0.2-1× SSC at 50° C. for high stringency hybridization conditions. Depending on the desired hybridization conditions, one may also vary the temperature of the wash step. For example, for low stringency hybridization one may wash at about room temperature (about 20-25° C.), for hybridization of moderate stringency one may wash at about 35-45° C. and for high stringency conditions at about 55-65° C.

In certain embodiments, polynucleotides may have sequences different from the naturally-occurring nucleic acid sequence in view of the redundancy in the genetic code, especially if the amino acid sequences are known. Various codon substitutions may be introduced to produce various restriction sites or to optimize expression in a particular system (e.g., codon usage of a cell).

4(A)(4) Other Cell Surface Proteins as Markers

In addition to receptors that render a cell susceptible to infection by viruses or phages, other polynucleotides encoding other cell surface proteins can be fused to screened polynucleotides to detect the presence of a polynucleotide encoding a cleavable N-terminal signal sequence. Any cell surface protein that can be detected in any manner may be employed. Examples include cell surface proteins that are needed for a cell to uptake a particular molecule such as a nutrient. Such cells can be cultured in media that includes such a molecule. The system is designed so that cells that include a sufficient amount of the given cell surface protein grow or survive better than cells that do not include a sufficient amount of the cell surface protein. One uses cells that do not include a sufficient amount of cell surface protein unless the polynucleotide encoding it is fused to screened polynucleotide that encodes a cleavable N- terminal signal sequence. As discussed above, that can be accomplished by using polynucleotide encoding the cell surface protein that lacks a sequence encoding a cleavable N-terminal signal sequence, which is fused to screened polynucleotide.

Known examples of such cell surface proteins include ScrY (sucrose transport), BtuB (vitamin B₁₂), FadL (fatty acids), LamB (maltose), and lutA (iron).

One may also employ cell surface proteins that can be detected by other methods. For example, one can use cell surface proteins that can be detected by the interaction of a ligand, such as an antibody, at the cell surface. Thus, one can detect whether the cell surface protein has been secreted and included at the cell surface by a subsequent binding of such a ligand.

4(A)(5) Other Elements of the Screening Cassettes

Screening cassettes useful for the described methods, in certain embodiments, contain additional elements. For example, the cassette may contain a promoter to direct the transcription of the ORF in a prokaryotic cell. Or, for example, the cassette may contain sequence elements to facilitate the translation of a messenger RNA transcription from the ORF of the screening cassette.

Promoters useful for the screening cassettes of the invention are preferably capable of facilitating transcription in a prokaryotic cell. Useful promoters include, but are not limited to, inducible promoters, constitute promoters, naturally occurring promoters, non-naturally occurring promoters, etc.

Examples of promoters useful for the screening cassettes of the invention are described, for example, by De Vries et al., 1984, Proc. Natl. Acad. Sci. USA 81:6080-6084. Further examples of useful promoters include, but are not limited to, the beta-lactamase and lactose promoter systems (Chang et al., 1978, Nature 275:615; Chang et al., 1987, Nature 198:1056; Goeddel et al., 1979, Nature 281:544), the arabinose promoter system (Guzman et al., 1992, J. Bacteriol. 174:7716-7728), alkaline phosphatase, a tryptophan (trp) promoter system (Goeddel et al., 1980, Nucl. Acids Res. 8:4057; Yelverton et al, 1981, Nucl. Acids Res. 9:731; U.S. Pat. No. 4,738,921; E.P.O. Pub. Nos. 36,776 and 121,775), and hybrid promoters such as the tac promoter (deBoer et al., 1983, Proc. Natl. Acad. Sci. USA 80:21-25), the beta.-lactomase (bla) promoter system (Weissmann, “The Cloning of Interferon and Other Mistakes” in Interferon 3 (ed. I. Gresser, 1981)). Bacteriophage lambda PL (Shimatake et al., 1981, Nature 292:128) and T5 (U.S. Pat. No. 4,689,406) promoter systems also provide useful promoter sequences.

Examples of non-naturally occurring promoters are synthetic hybrid promoters comprising sequences from promoter or non-promoter polynucleotides as described in U.S. Pat. No. 4,551,433; Studier et al., 1986, J. Mol. Biol. 189:113; Amann et al., 1983, Gene 25: 167; de Boer et al., 1983, Proc. Natl. Acad. Sci. 80:21; E.P.O. Pub. No. 267,851; Tabor et al., 1985, Proc Natl. Acad. Sci. 82:1074.

In certain embodiments, the screening cassette contains a Shine-Dalgamo (SD) sequence (Shine et al., 1975, Nature 254:34) to promote binding of mRNA to the ribosome through hybridization of bases in the SD sequence and the 3′ and of E. coli 16S rRNA (Steitz et al., “Genetic signals and nucleotide sequences in messenger RNA” in Biological Regulation and Development: Gene Expression (ed. R. F. Goldberger, 1979)).

In certain embodiments, a promoter useful in the screening cassette of the invention contains an operator domain. The operator domain may overlap with an adjacent RNA polymerase binding site at which RNA synthesis begins. A gene repressor protein may bind the operator and thereby inhibit transcription. Constitutive expression may occur in the absence of the repressor protein. Or, a gene activator protein may bind the operator to stimulate transcription. The catabolite activator protein (CAP) is a gene activator protein which stimulates ranscription of the lac operon in E. coli (Raibaud et al., 1984, Annu. Rev. Genet. 18:173). Thus, an operator domain may function either to inhibit or to stimulate transcription.

In certain embodiments, transcription termination sequences are included in the screening cassette of the invention. Preferably a transcription termination sequence is located 3′ to the translation stop codon and therefore flanks the coding sequence together with the promoter. Transcription termination sequences frequently include DNA sequences (of about 50 nucleotides) which can form stem loop structures. Examples include transcription termination sequences derived from genes with strong promoters, such as the trp gene in E. coli as well as other biosynthetic genes.

4(A)(6) Vectors

In certain embodiments, the polynucleotides used in the described methods are part of a vector. A vector may include, for example, the screening cassette discussed above. The vector may facilitate the maintenance in a prokaryotic cell of the polynucleotides used in the described methods, for example, the screening cassette. Vectors that may be used include, but are not limited to, extrachromosomal and intrachromosomal vectors, i.e., vectors that do not integrate into the host cell genome and vectors that do integrate.

In certain embodiments, a vector useful for the described methods contains a selectable marker to identify cells that have taken up the vector. A selectable marker may provide a growth advantage to the host cell. Alternatively, it may help to identify the host cell through a color indicator (e.g., a dye or a fluourescent marker). Selectable markers that can be used include, but are not limited to, genes that confer resistance to drugs such as ampicillin, erythromycin, chloramphenicol, kanamycin (neomycin), and tetracycline (Davies et al., 1978, Annu. Rev.Microbiol. 32:469). Biosynthetic genes, for example, a gene in the histidine, tryptophan, and leucine biosynthetic pathways, may also be used as a selectable marker that provides a growth advantage under appropriate culture conditions.

A variety of vectors have been developed for transformation into many bacteria. Such vectors include, but are not limited to, commercially available vectors set forth in catalogs of Stratagene (La Jolla, Calif.), Novagen (Madison, Wis.), and InVitrogene (Carlsbad, Calif.).

4(B) Proteins and Polynucleotides Encoding such Proteins that can be Identified Using the Described Methods

In preferred embodiments, the described methods are useful for identifying proteins that contain an amino acid sequence which may function as a cleavable N-terminal signal sequence in a prokaryotic cell. Proteins which contain a cleavable N-terminal signal sequence typically fall into a number of classes which are of unique interest for therapeutics and diagnostics.

4(B)(1) Eukaryotic Proteins and Polynucleotides

A variety of known eukaryotic proteins contain cleavable N-terminal signal sequences including, but not limited to, hormones, growth factors, membrane receptors, secreted proteins, receptor kinases, etc. The methods of the invention can be used to identify new members of any of these protein families. In addition, the methods of the invention can be used to identify new families of proteins with a cleavable N-terminal signal sequence.

4(B)(2) Prokaryotic Proteins and Polynucleotides

Prokaryotic organisms express a variety of proteins with a cleavable N-terminal signal sequence including, but not limited to, secreted proteins, membrane receptors, transport proteins, and periplasmic enzymes. Many of these proteins are involved in the pathogenic effect that the prokaryotic organisms exert in higher organisms, including humans. Many of these proteins may also be used to detect particular prokaryotic organisms or strains of orgnisms in diagnostic procedures.

4(B)(3) Invasins

The pathogenic response elicited by many bacteria when infecting mammalian cells involves the invasion of the host cells by cytotoxic proteins called invasins that are encoded by the bacteria. See, Cornelius, 1998, J. Bacteriol. 180:5495-5504, which discusses invasins. The identification of invasins with the methods of the invention will aid in the development of therapeutics to neutralize these toxins. Invasins are secreted by pathogenic bacteria following a signal generated by their close proximity to the mammalian cell (Cornelius, 1998, supra). The signal is transmitted via a membrane receptor that is present on the surface of the bacteria. The invasins may be identified with the described methods, as well as, the membrane receptors.

Invasins are secreted by the Type III secretory apparatus, which typically involves a number of host-encoded proteins and does not result in the N-terminal cleavage of the protein to be exported. See, Hueck, 1998, Micro. Mol. Biol. Rev. 62:379-433, which discusses mechanisms of protein secretion. The Type III secretion system is used by pathogenic bacteria to extrude cytotoxic invasins into sensitive cells to elicit the pathogenic response (Faruque et al., 1998, Micro. Mol. Biol. Rev. 62:1301-1314). Examples of such pathogenic bacteria are Yersinia, Salmonella, Vibrio (Hueck, 1998, supra; Faruque et al., 1998, supra; Mecsas et al., 1991, Emerg. Infect. Dis. 2:271-288; Finlay et al., 1997, Micro. Mol. Biol. Rev. 61:136-169; Galan, 1996, Mol. Micro. 20:263-271).

Although invasins are not N-terminally processed, it has been demonstrated that an N- terminal sequence stretch of invasins has the characteristics that allow secretion of these proteins (Anderson et al., 1997, Science 278:1140-1143). The N-terninal sequences of invasins do not contain a consensus sequence that can be readily identified by sequence analysis. Furthermore, it has been shown that if the N-terminal segment of a Type III secreted protein is fused to a second polypeptide that is not secreted in this manner, the N-terminus can direct the hybrid protein to be secreted by the Type III pathway (Michiels et al., 1991, J. Bacteriol. 173:1677-1685).

Type III secretion typically involves the presence of host proteins in the secreting cell. Therefore, the described methods are preferably used in a prokaryotic cell that expresses these host proteins to facilitate Type III secretion. Examples of such prokaryotes include, but are not limited to, Yersinia, Salmonella, Vibrio. In certain embodiments, polynucleotides encoding proteins involved in Type III secretion can be expressed in a prokaryotic cell that does not naturally express those proteins.

4(C) Libraries that can be Screened

Any type of polynucleotide library can be screened to identify proteins with a cleavable N-terminal signal sequence using the described methods. Examples of libraries that can be screened with the methods of the invention include, but are not limited to, cDNA libraries and genomic libraries. See, for example, Sambrook et al., 1989, supra; and Ausubel et al., 1989, supra, which discuss different types of polynucleotide libraries and methods of preparing such libraries.

In certain embodiments, a library screened with the disclosed methods is prepared using a method that increases the likelihood that polynucleotide sequences encoding cleavable N-terminal signal sequences are presented in the library. These sequences are typically found in the 5′ region of an mRNA molecule and, therefore, a preferred library includes many polynucleotide clones that correspond to the 5′ region of mRNA molecules. A variety of techniques are known in the art of biotechnology to prepare polynucleotide libraries that include a high percentage of polynucleotide clones that correspond to the 5′ region of mRNA molecules. These techniques include, but are not limited to, the RACE (Rapid Amplification of cDNA Ends) technique. RACE is a proven PCR-based strategy for amplifying the 5′ end of cDNAs. 5′-RACE-Ready cDNA synthesized from human fetal liver containing a unique anchor sequence is commercially available (Clontech). See also, Bertling et al., 1993, PCR Methods and Applications 3:95-99, which discusses the RACE method.

Libraries prepared from any organism can be screened with the disclosed methods. For example, libraries prepared from a eukaryotic or prokaryotic organism may be screened. In certain embodiments, when a library is prepared from a eukaryotic organism, a cDNA library is preferred. In certain embodiments, when a library is prepared from a prokaryotic organism, a genomic library is preferred.

Libraries prepared from a eukaryotic organism may be prepared from any organ, tissue or cell line. Tissues from which a library may be made include, but are not limited to, glands, adrenal gland, mammary gland, pituitary gland, thymus gland, thyroid gland, pankreas, prostate, testis, brain, amygdala, caudate nucleus, cerebellum, hippocampus, substantia nigra, subthalamic nucleus, thalamus, frontal lobe, spinal cord, sciatic nerve, bone marrow, spleen, placenta, small intestine, heart, kidney, tonsil, lung, trachea, lymph node, uterus, skeletal muscle, smooth muscle, epithelia, connective tissue, etc. Cell lines from which a library may be made include, but are not limited to, primary cell lines, secondary cell lines, transformed cell lines, NIH3T3 cells, HeLa cells, mouse L cells, COS cells, COS 7 cells, CHO cells, 293 cells, Jurkat cells, or any other cell line deposited with and available from the American Type Culture Collection, Maryland, USA.

Libraries screened with the disclosed methods may be prepared from a fungus including, but not limited to, Candida albicans, Aspergillus fumigatus, Microsporum spp., Blastomyces dermatitidis.

Libraries screened with the disclosed methods may be prepared from a bacteria including, but not limited to, pathogenic bacteria, animal pathogenic bacteria, plant pathogenic bacteria, Vibrio cholerae, Erwinia amylovaria, Yersinia pestis, Pseudomonas syringae, Salmonella, Xanthomonas campestris, Shigella, Ralsortia solanacearum, E. coli, enteropathogenic E. coli, Pseudomonas aeruginosa, Chlamydia psittaci, Yersinia, Salmonella, Vibrio.

In certain embodiments, polynucleotides that are not part of a library may also be screened using the described methods.

4(C)(1) Size Selection of Libraries

The methods of the invention facilitate the screening for polynucleotides that correspond to proteins with a cleavable N-terminal signal sequence. In certain embodiments, the screened polynucleotides are from about 100 base pairs to about 600 base pairs in length. Thus, a library of polynucleotides screened using the described methods preferably comprises a large percentage of polynucleotides in, or close to, the preferred size range.

A variety of methods to size select polynucleotide libraries are known in the field of biotechnology. These methods include, but are not limited to, gel electrophoresis, column chromatography, restriction endonuclease digestion with a frequently cutting enzyme (e.g., an enzyme with a short recognition sequence, for example, four nucleotides). See, for example, Sambrook et al., 1989, supra; and Ausubel et al., 1989, supra, which discuss techniques useful to size select polynucleotide libraries.

4(D) Prokaryotic and Archaebacterial Host Cells

Any prokaryotic cell can be used as a host cell in the methods of the invention. These host cells include, but are not limited to, bacteria, gram positive bacteria, gram negative bacteria, enteric bacteria, and E. coli, etc. One may also employ archaebacteria as the host cells.

In a preferred embodiment, the prokaryotic cells used in the described methods should not be amenable to infection by the phage or virus that is used to identify the desired polynucleotide clones. For example, and not by way of limitation, if the lamB protein is used as the marker in the screening cassette and if lambda phage is used to screen for the expression of lamB protein, then the host cells should not be susceptible to lambda phage infection to a degree that would make it impossible to identify desired cells (i.e., cells that express a lamB fusion protein with a cleavable N-terminal signal sequence expressed from the screening cassette). An example of such host cells are XLOLR E. Coli cells (Stratagene, Calif., USA).

4(D)(1) Introducing the Screening Cassette into the Host Cells

Any method known in the art of biotechnology can be used to introduce the screening cassette in the host cells used in the described methods. These methods include, but are not limited to, treatment of the cells to render them competent to take up polynucleotides and electroporation. For example, cells can be exposed to CaCl₂ or other agents, such as divalent cations and DMSO or electroporation.

Transformation procedures may vary depending on the bacterial species to be transformed. See, e.g., Miller et al., 1988, Proc. Natl. Acad. Sci. 85:856; Wang et al., 1990, J. Bacteriol. 172:949, which discuss the transformation of Campylobacter. See, e.g., Masson et al., 1989, FEMS Microbiol. Lett. 60:273; Palva et al., 1982, Proc. Natl. Acad. Sci. USA 79:5582, which discuss the transformation of Bacillus. See, e.g., Chassy et al., 1987, FEMS Microbiol. Lett. 44:173, which discusses the transformation of Lactobacillus. See, e.g., Cohen et al., 1973, Proc. Natl. Acad. Sci. 69:2110; Dower et al., 1988, Nucleic Acids Res. 16:6127; Kushner, “An improved method for transformation of Escherichia coli with ColE1-derived plasmids” in Genetic Engineering: Proceedings of the International Symposium on Genetic Engineering (eds. H. W. Boyer and S. Nicosia, 1978); Mandel et al., 1970, J. Mol. Biol. 53:159; Taketo, 1988, Biochim. Biophys. Acta 949:318, which discuss the transformation of Escherichia. See, e.g., Barany et al., 1980, J. Bacteriol. 144:698; Harlander, “Transformation of Streptococcus lactis by electroporation,” in Streptococcal Genetics (ed. J. Ferretti and R. Curtiss III, 1987); Perry et al., 1981, Infec. Immun. 32:1295; Powell et al., 1988, Appl. Environ. Microbiol. 54:655; Somkuti et al., 1987, Proc. 4th Evr. Cong. Biotechnology 1:412, which discuss the transformation of Streptococcus. See, e.g., Fiedler et al., 1988, Anal. Biochem 170:38, which discusses the transformation of Pseudomonas. See, e.g., Augustin et al., 1990, FEMS Microbiol. Lett. 66:203, which discusses the transformation of Staphylococcus.

Other transformation procedures that may be used are described in U.S. patent application Ser. No. 60/146,516, filed Jul. 30, 1999, and Ser. No. 09/253,703, filed Feb. 22, 1999.

4(E) Cloning Full-length Polynucleotide Sequences

In certain embodiments, the polynucleotides identified using the methods of the invention only represent a part of a mRNA or a gene. In order to obtain the entire sequence of the mRNA, or its corresponding cDNA, or a gene, one may isolate a full-length clone or one or more partial clones to obtain the missing sequence information. The polynucleotides isolated using the described methods can be used, for example, as hybridization probes, to obtain further polynucleotide clones corresponding to the gene or cDNA of interest.

Full-length clones or further partial clones can be isolated, for example, by screening a library. In certain embodiments, a library that contains full-length clones is screened with the polynucleotide identified with the described methods. See, for example, Sambrook et al., 1989, supra; and Ausubel et al., 1989, supra, which discuss the preparation of cDNA and genomic libraries that are likely to contain full-length clones. In certain embodiments, the library that is screened is prepared from an organism, tissue, organ and/or cell line that corresponds to the organism, tissue, organ and/or cell line from which a polynucleotide identified with the methods of the invention was obtained. In certain embodiments, more than one library is screened to obtain the entire desired sequence information.

4(F) Uses of Identified Polynucleotides and Proteins

Polynucleotides encoding a cleavable N-terminal signal sequence in a bacterial genomic library likely encode bacterial surface proteins. Identification of such proteins allows one to design therapeutics or other antimicrobial agents, such as antiseptics, that act on such surface proteins, and thus, may be useful against pathogenic bacteria. Also, such proteins may be useful for diagnosing the presence of a particular organism. Since the proteinS are on the cell surface, antibodies or molecules that behave as antibodies (e.g., aptamers) may be used to identify cells with such proteins on their surface. The polynucleotides may be used to design diagnostic probes that can be used to detect the presence of a particular organism that includes such polynucleotides.

Polynucleotides encoding a cleavable N-terminal signal sequence in mammalian cells, often encode cell surface receptors. Such receptors can be used to screen for molecules that stimulate or activate such receptors. Often such stimulation or activation results from a molecule that binds to the cell surface receptors.

### 5.0 EXAMPLES

### 5(A) Example

### Identification of Eukaryotic Proteins with a Cleavable N-terminal Signal Sequence Preliminary Results

1. Mammalian Signal Sequences Function in E. coli

Evidence in the literature suggests that signal peptides from mammalian genes function as signal peptides in E. coli. (Zheng et al., 1996, Cell 86:849-852). The lamB selection system confirmed that suggestion. In the E. coli strain XLOLR (lamB⁻) (Stratagene, Calif., USA), expression of LamB on a colE1 origin plasmid resulted in restoration of lambda infectibility in the XLOLR strain. Specifically, the gene encoding lamB was inserted into the pKK223-3 plasmid (pKK223-3 plasmid is available from ClonTech (Palo Alto, Calif.)).

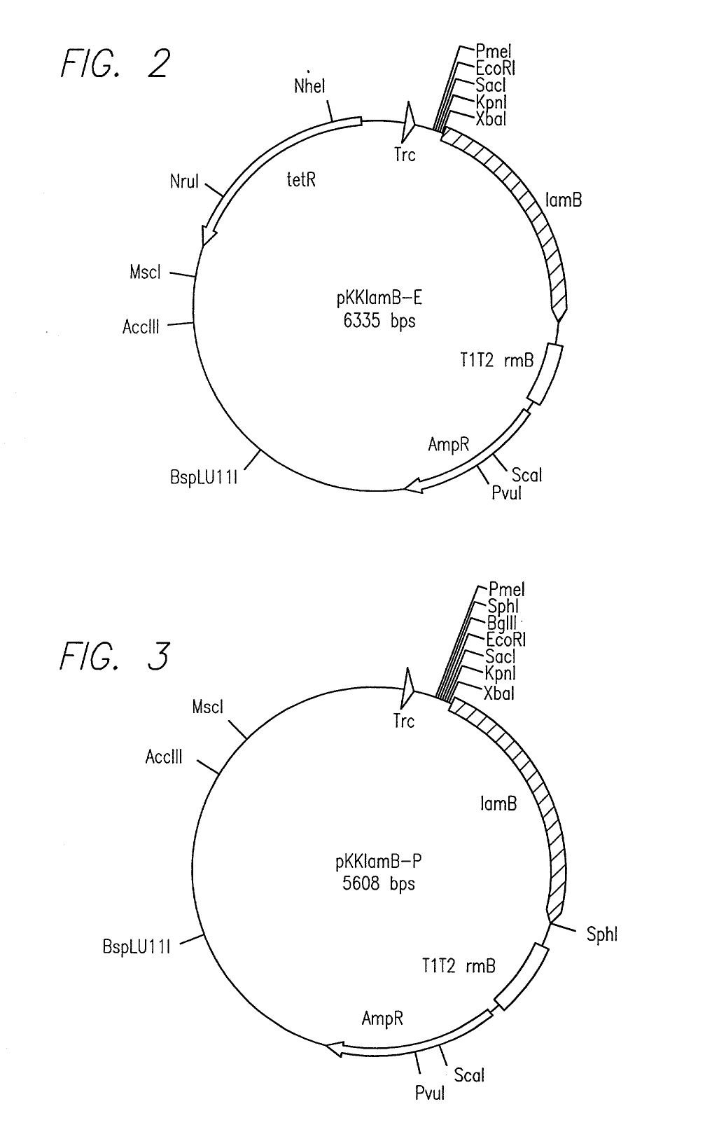

When nucleotides encoding the signal peptide were removed from the plasmid-encoded lamB gene, lambda phage did not infect the cells as judged by the lack of lambda lysogeny using the gt10 Kan^{R} virus. For this work, the lamB gene lacking nucleotides encoding the signal peptide were included in the pKK223-3 plasmid, which resulted in the pKKLamB-E plasmid, which is depicted in FIG. 2. The particular λgt10(KanR) virus was constructed at Stratagene and it efficiently lysogenizes E. Coli when a lamB receptor is included on the surface of the cells. Any other similar virus-that may be constructed by one skilled in the art that efficiently lysogenizes E. coli may be used.

Polynucleotides encoding the N-terminal signal peptides from preprotrypsin, T cell growth factor α (TGFα) (tgf is transforming growth factor) (Brachmann et al., 1989, Cell 56:691-700), epidermal growth factor receptor (EGFR) (Tang et al., 1997, J. Biochem. 122:686-690), and the HER2 receptor (Natali et al., 1990, Int. J. Cancer 45:457-461), were inserted directly upstream of the lamB gene lacking its signal peptide. This was accomplished by PCR amplification of the appropriate sequences in the pkkLamB-E vector. When these plasmids were introduced into XLOLR cells, they were lambda infectible. These data suggested that many or most eukaryotic signal peptides would function in the same capacity in E. coli cells and showed that selection for signal peptides from eukaryotic cDNA libraries in E. coli was possible.

2. The LamB Receptor Activity may be Sensitive to Large Additions

Fusion proteins comprising the LamB protein and a segment of a mature N-terminus of another protein preceding LamB allowed LamB to function as a viral receptor. Genetic fusions between the signal peptide-less lamB and either the TGFα or EGFR receptor genes were made using increasingly larger segments of the mammalian genes. When the signal peptide was followed by the N-terminal 37 or 434 amino acids of EGFR, these fusions gave rise to lambda infectible XLOLR cells. When the entire 675 amino acid coding region of EGFR preceded LamB, XLOLR cells were not lambda infectible. When the N-terminal 27 amino acids of TGFoc growth factor gene was fused to LamB, XLOLR cells containing this plasmid were lambda infectible. A fusion of the entire TGFA coding region (152 amino acids) resulted in noninfectible XLOLR cells. These results demonstrate that if a signal trap library is constructed and screened in a lamB vector, smaller cDNA fragments may be preferable to avoid the potential for inhibition of LamB activity.

3. Screening Eukaryotic cDNA Libraries with the pKKLamB-E Vector

Based on the initial results in Sections 5(A)(1) and (2), cDNA libraries (bovine brain and rat brain) were cloned into the EcoRI site of the pKK LamB-E selection vector (FIG. 2) was screened. A randomly primed cDNA library from rat brain (purified by fractionation of total RNA on an oligo-dT column) was size selected by size exclusion chromatography for fragments 100-600 bp. EcoRI adapters were ligated to the cDNA ends and cloned into EcoRI digested pKK LamB-E.

The ligation mix was transformed into XL10-Gold (Stratagene, Calif., USA) and a library of approximately 4×10⁵ primary clones was obtained. See Stratagene XL1 Blue Competent Cell Manual. Although this number was significantly lower than anticipated, the library was screened in order to evaluate whether a complex mixture of plasmids could be screened to obtain mammalian genes containing a signal peptide coding region. The transformant colonies were pooled, plasmid DNA purified, and the library transformed into XLOLR (lamB⁻). See Stratagene XLI Blue Competent Cell Manual. The initial transformation into XL10-Gold was performed to increase the primary library size. It was observed that entry of ligated DNA into XL10-GOLD is significantly better than certain other chemically competent E. coli hosts (Jerpseth et al., 1997, Strategies 10:37-38). However, this double round of amplification may pose a problem in achieving complete and representative libraries due to clonal growth bias.

The XLOLR transformed cells containing the pKK LamB-E cDNA library were pooled and subjected to λgt10 Kan^{R} infection under conditions favoring the lysogenic pathway (infection, expression, and plating at 30° C.). One hundred (100) μl of logarithmic XLOLR cells were infected with approximately 10⁸ λgt10(Kan^{R}) phage. The cells and phage were incubated at 30° C. for 30 minutes (not shaking). Then, 0.5 ml LB media was added, the cells were grown for 90 minutes at 30° C., and were plated on LBKan plates at 30° C. A total of approximately 250 colonies were isolated.

Ninety-six (96) of these were miniprepped and retransformed back into XLOLR for confirmation. Ninety (90) of the 96 clones tested maintained their lambda infectible phenotype as assayed by lambda gt10 Kan^{R} colony formation or plaque formation when a lytic lambda virus was used. The later test with lytic lambda virus is a screen to assure that the cells actually included lamb on the cell surface, which confirms whether a mammalian signal sequence is included in the pKK LamB-E vector.

After a lambda phage infects a cell, a gene carried by the lambda phage that encodes a lambda repressor molecule is expressed by the cell to produce the lambda repressor. The presence of the repressor molecule in the cell prevents a subsequent lambda phage from replicating in the cell. It is possible that cells were spontaneously kanamycin resistant in these described procedures without having lamB on their surface. To exclude the possibility of such mutants or contaminants, one can test for the presence of lamB on the surface with a special lambda phage that is capable of infecting cells that include lamB on the surface and that already are lysogenic for lambda phage. Such lambda phages can replicate in the presence of the repressor molecule that typically would prevent a subsequent lambda phage from replicating in a cell that has already lysogenic for lambda phage. In this experiment, the lambda phage L2B was used to infect KanR colonies. L2B is able to replicate in and lyse E. coli cells that already include lambda lysogens (in this case the lambda lysogen is the lambda gt10 Kan^{R}). However, other phages that can infect cells having lamB on their surface and that are already infected by a lambda phage are known in the art and can be used. See, e.g, Hendrix et al., 1983, Lambda II, Cold Spring Harbor Press, Cold Spring Harbor, N.Y.

XLOLR cells carrying the pKK LamB vector without an insert never yielded KanR colonies or plaques.

Sixteen of the positive clones were then sequenced to determine the identity of the inserted segment. Two of the 16 corresponded to known membrane receptor or secreted proteins. Both of these inserts included the putative signal peptides of the mammalian protein. They were the paranodin receptor (Menegoz et al., 1997, Neuron 19:319-331) and the tissue-specific inhibitor of metalloprotease 2 (DeClerck et al., 1992, Genomics 14:782-784). One of the 16 isolates was present in the NIH database but no function was ascribed. Six of the 16 inserts were not present in the NIH database, indicating they may correspond to unknown genes. Finally, seven of the 16 were observed to be 28S rRNA genes (different segments) that had been converted to cDNA.

The finding that paranodin receptor (Menegoz et al., 1997, Neuron 19:319-331) and tissue-specific inhibitor of metalloprotease TIMP2 (DeClerck et al., 1992, Genomics 14:782-784) were isolated in this system validated the selection. The unknown sequences (6 of 16) indicate that this system may be a rapid method to clone new genes or discover 5′ coding regions of 3′ ESTs in the database.

### 5(B) Example

### The pKK LamB-P Vectors

For screening nucleotides encoding prokaryotic proteins, the the pKK LamB-E vector was modified to include additional multiple cloning sites directly upstream of the lamB gene to produce three versions (1, 2, and 3) of pKK LamB-P (See FIG. 3). The multiple cloning sites provide for the three reading frames. Although the presence of the unique EcoRI site may be sufficient for screening cDNA libraries, it is intended that this vector be amenable to screen genomic libraries of small fragments. Therefore, an oligonucleotide is inserted with unique restriction sites for BglII and Sphl to lie immediately upstream of the EcoRi site. These sites are used for Sau3A and NlaIII genomic libraries (the EcoRi site is used in conjunction with Tsp509I) (Tsp509I is a restriction endonuclease commercially available from New England BioLabs (Beverly, Mass.) The ability to create libraries with a few different enzymes is important to optimize the identification of large numbers of signal sequence clones regardless of the presence or absence of particular restriction sites in the vicinity (<1 kb) of the signal sequence. Since translational fusions to pKK LamB-P are made, three separate versions of this plasmid are created, i.e., one for each of the three reading frames. This ensures that for any particular restriction digest used to make the genomic library, signal peptide encoding regions would not be lost due to incorrect reading frame fusions with pKK LamB-P.

### 5(C) Example

### Transformation of E. Coli

1. Background

There are at least three major advantages to an E. coli system for selecting cDNAs encoding secreted proteins and membrane receptors. They are the size of libraries that can readily be screened, the speed at which the research can proceed, and the potential to eliminate bias that occurs when libraries destined for a eukaryotic host must first be amplified once or twice in E. coli. It has long been a concern of researchers screening libraries that E. coli selectively allows certain clones to replicate better than others resulting in a pool of molecules that is not necessarily proportional to the cDNA starting pool. For example, the inventors found that cDNAs that encode toxic genes, certain membrane proteins or DNA binding proteins are particularly prone to be replicated poorly by the bacterial host. Thus, after a number of generations of growth to prepare plasmid DNA, some molecules are selectively lost and are underrepresented in the population. This may be related to their abundance in the mRNA population or it may result from a growth bias in XL10-GOLD prior to introduction into XLOLR. The intermediate transformation into XL10-GOLD was performed because an improved transformation efficiency of ligated DNA molecules using this strain was observed. However, this potential advantage may be offset by the need for extensive growth of the library in E. coli prior to the functional selection.

2. Transformation if E. coli

To address this concern of a potential clonal bias, the cDNA library ligation is directly introduced into the XLOLR selection strain. According to certain embodiments, one can employ an E. coli electroporation procedure disclosed in U.S. patent application Ser. No. 09/253,703, filed February 22, 1999, and Ser. No. 60/103,612, filed Oct. 9, 1999, and in PCT Application No. PCT/US99/23216, filed Oct. 6, 1999. These applications are incorporated by reference herein. That procedure improves electroporation of E. coli cells. Efficiencies for ligated DNA transformation via electroporation were achieved that are comparable to XL10-GOLD. This speeds up the entire selection process and eliminates a potential source of unnecessary amplification.

The paranodin and TIMP2 clones obtained above are used (i.e., as positive lamB plasmids) to monitor the effect of altering these conditions. Electroporation of XLOLR cells is carried out with a mixed population of starting plasmid DNA (i.e., a library of polynucleotides inserted into a screening plasmid) and the positive lamB plasmid, using ratios that vary from 1 :1 to 10⁶:1. The minimum time of growth required to retrieve the positive clone is ascertained at each step and its proportion among the total number of transformants is determined and tabulated separately. Although the best test is a true library ligation, the use of purified plasmid DNA clones can help define the optimal parameters.

### 5(D) Example

### Retrieval of Full-length cDNA from a Signal Peptide Encoding Polynucleotide Fragment

The signal trap method to clone DNA segments results in obtaining the 5′ end of the coding region of each gene, including the ATG translational start. In traditional cDNA libraries that are primed using an oligo-dT strategy (to ensure that the 3′ poly A sequence is present) many (or most) cDNA clones are not full length but represent the 3′ end of the gene. This results since reverse trancriptase, the enzyme responsible for first strand synthesis off the mRNA template, does not always traverse the entire mRNA due to either a lack of processivity or particular sequences that create a secondary structure that blocks its progress. Therefore, many genes present in the NIH database lack the 5′ end. In these instances, researchers use the 3′ ends of genes to retrieve the full length cDNA by a variety of strategies. In the signal trap method, the situation is reversed. In other words, the 5′ end is cloned and it is used to isolate the entire cDNA of the corresponding gene. This can be accomplished by using the 3′ RACE Kit of ClonTech (Bertling et al., 1993, PCR Methods and Applications 3:95-99). This method is commonly used for this purpose and is very amenable for doing multiple clones simultaneously. DNA corresponding to the entire open reading frame of each 5′ segment is retrieved.

### 5(F) Example

### Screening Human cDNA Libraries

Using the pKK LamB-E shown in FIG. 2, one can screen a cDNA library. The screened cDNA libraries can be prepared from any human tissue or organ, including but not limited to, human liver, whole brain and skeletal muscle.

To screen the library and minimize potential amplification bias, one can use the method described above for immediate selection in Example 5(D)2. The cDNA ligation reaction is electroporated into XLOLR. After the minimal post shock expression, the transformants are infected with λgt10 Kan^{R} at 30° C. for 30 minutes. Additional growth at 30° C. (minimum) is provided to allow for expression of the Kan resistance protein. This mix is then directly plated onto kanamycin +ampicillin plates to select for XLOLR transformants that have been infected by λgt10 Kan^{R}.

In order to rapidly verify that the colonies obtained are true positives, the following procedure is carried out. Small cultures (100 ul) of each potential positive are grown to stationary phase and infected with a lambda phage (λ: 1098) that carries the Tet resistance gene flanked by IS10 insertion sequence ends and also carries the transposase specific for IS10 (See Kleckner et al., 1991, Methods Enzymol. 204:139-180) Cells that are lambda lysogens do not serve as hosts for productive superinfection by an identical phage by virtue of its synthesis of the repressor (cI) protein. (Hendrix et al., 1983, Lambda II, Cold Spring Harbor Press, Cold Spring Harbor, N.Y.). However, the λ:1098 is capable of entering a lysogenic cell if a LamB receptor is present on the surface. The λ:1098 will not enter a lysogenic cell if a LamB receptor is not present on the surface. Thus, if the screened polynucleotide encodes a cleavable N-terminal signal sequence, a lysogenic cell can be infected by λ:1098 and therefore can be rendered Tet resistant. If the screened polynucleotide does not encode a cleavable N-terminal signal sequence, the cell will not be infected by λ:1098.

The λ:1098 phage was specifically disabled so that it cannot replicate, integrate, or produce lysis proteins in an E. coli host that does not carry a particular tRNA suppressor (for example, XLOLR is such a strain) (Kleckner et al., 1991, Methods Enzymol. 204:139-180). Once the lambda DNA enters the cell, it retains its ability to express the transposase and allow the TetR gene to transpose randomly into chromosomal or plasmid DNA. The efficiency of this process is 10-4 per infected cell (Kleckner et al., 1991, Methods Enzymol. 204:139-180). However, if the pKK LamB plasmid produces a functional lamB receptor, this gives rise to >100 Tet^{R} colonies under these conditions. Thus, when polynucleotides encoding cleavable N-terminal signal sequence are screened, an increase in the number of Tet^{R} colonies is observed. Any true positive isolated in the Kan^{R}+Amp^{R} selection should give rise to Tet^{R} colonies upon λ:1098 infection. Plasmid DNA from all clones that have been selected and screened in this manner are purified and sequenced. Each clone is analyzed by database comparisons. Each unique clone (known or unknown) is saved to serve as a probe for retrieving the full length coding region.

### 5(F) Example

### Screening cDNA Libraries from Fungi

An additional application of the signal trap system to the development of therapeutics and diagnostics is in the study of human, animal, and plant pathogens. These agents may be eukaryotic (for example, fungi) or prokaryotic (for example, pathogenic bacteria). The system outlined in the previous sections can be readily used to identify pathogens from eukaryotes like fungi. cDNA libraries from a number of fungal organisms are made, for example, Candida albicans, Aspergillus fumigatus, Microsporum spp., Blastomyces dermatitidis. These organisms were chosen for both their medical relevance and to further validate the selection system. Normalized cDNA libraries from these organisms are constructed and screened as detailed above. Positive clones are sequenced and used as probes to isolate full length cDNA.

### 5(G) Example

### Screening Bacterial Genomic Libraries

1. Background

An advantage of a bacterial signal trap system over a eukaryotic system is its versatility to analyze genomic libraries of prokaryotic organisms. Prokaryotic expression libraries typically cannot be effectively screened in eukaryotic cells. The presence of ATG codons that may lie upstream of the translational start in the prokaryotic DNA (whether in frame or not) may divert the eukaryotic translational machinery to an incorrect site and result in no expression of the desired coding region even if a eukaryotic promoter was present. Lewin, in Genes V (1998) Cell Press, Cambridge, Mass.

Discovery of secreted proteins and membrane receptors from eubacteria have direct antimicrobial applications, including therapeutic and antiseptic applications, particularly for the identification of surface targets on pathogenic bacteria. The screening system described herein can be applied to the ecological control of microorganisms by identifying cell surface receptors that could serve as targets for molecular intervention. In addition, analysis of bacterial proteins that are membrane bound and secreted are also important to many basic science researchers in their efforts to more completely understand prokaryotic signal transduction. Eukaryotic systems typically cannot be used for this purpose.

Prokaryotic organisms secrete proteins by at least 4 distinguishable mechanisms (Hueck, 1998, Micro. Mol. Biol. Rev. 62:379-433; Wandersman (1996) in Esherichia coli and Salmonella, 955-966, 2^{nd} ed, ASM Press, Washington, D.C.). Two of these require that an N-terminal cleavable signal peptide precede the mature protein and further typically require additional host proteins for extracellular secretion. These two systems are distinguishable by the additional host protein requirements. However, since both classes of proteins are synthesized as propeptides (with a standard N-terminal signal peptide) and since LamB does not need to be secreted extracellularly, these genes can be identified using the methods described herein.

The Type III secretion system is responsible for transporting many proteins that are the subject of investigation by pharmaceutical researchers and medical microbiologists. The Type III secretion system is used by pathogenic bacteria (e.g., Yersinia, Salmonella, Vibrio etc.) (Hueck, 1998, supra; Faruque et al., 1998, supra; Mecsas et al., 1991, Emerg. Infect. Dis. 2:271-288; Finlay et al., 1997, Micro. Mol. Biol. Rev. 61:136-169; Galan, 1996, Mol. Micro. 20:263-271) to extrude cytotoxic invasins into sensitive cells to elicit the pathogenic response (Faruque et al., 1998). These proteins carry an N-terminal sequence that is essential for their secretion, but the N-terminal sequence is not typically cleaved. It has been shown that a number of host proteins are involved in Type III secretion (Faruque et al., 1998). Some of these host proteins that are involved in Type III secretion are membrane bound and are also preceded by a cleavable N-terminal signal peptide. Invasins and accessory genes that aid in Type III secretion may be identified using the genomic library approach described herein.

2. Screening Bacterial Genomic Libraries

Genomic DNA of E. coli, Salmonella typhimurium, and Heliobacter pylori is prepared. These organisms were selected since retrieval and sequence determination of most or all identified polynucleotides should result in identification of the cloned gene. The genomic DNA from these bacteria are cleaved with either Sau3A (ligatable to ends created by BglII), Tsp509I (ligatable to EcoRI) (Tsp5O9I is available from New England BioLabs, Beverly, Mass). These restriction fragment libraries are ligated to the lamB vectors pKK LamB-P 1, 2, and 3 digested with the appropriate restriction enzyme containing compatible ends. Three lamB genomic cloning vectors are used, each with its multiple cloning site in a different reading frame relative to LamB. The three 4 base cutting enzymes are used to optimize the ability to obtain average size inserts of approximately 250 base pairs. Multiple enzymes reduce the chance of not retrieving some secreted genes because of a restriction site that falls within its signal peptide or lack of a nearby site resulting in a very long genetic fusion to lamB. Furthermore, because a functional translational fusion with LamB and each digestion results in only one reading frame, all three genomic vectors (i.e., one for each reading frame, or the 0, +1, and +2 vector) are prepared and used for ligation, and each genomic library ligation is pooled prior to electroporation into XLOLR. This helps ensure that each potential signal peptide can be in frame with LamB when the 3 ligation reactions are pooled and electroporated.

Since the average size fragment optimally is about 250 base pairs and bacterial genomic DNA is approximately 5000 kb, for each individual ligation, a 1× representation corresponds to 2×10⁴ inserts. However, since cloning is bidirectional, only 50% are in the correct orientation which doubles this number to 4×10⁴. Because the three ligations are combined into one pool for one library, 1× requires 1.2×10⁵ clones. To be confident that all genomic segments are represented, a 10× coverage, or a 1.2×10⁶ primary library size is the optimal target.

Each library that is electroporated into XLOLR is immediately infected with the λgt10 Kan^{R} phage and kanamycin resistant cells are selected. A total of 9 libraries are analyzed—a library made from fragments obtained following Sau3A, Tsp509I, or NlaIII digestion and each of the three in three reading frames. Positive clones are analyzed by DNA sequencing and database comparisons. Positive clones that are identified but whose function is unknown may provide clues to their biology.

3. Screening an E. coli Genomic Library

An E. coli genomic library was prepared by digesting total E. Coli genomic DNA with Tsp509I or Sau3Al restriction endonucleases and by ligating the fragments into the pKK LamB-P 1, 2, and 3 vectors that were digested with EcoRI (compatible with Tsp509I) or BamHI (compatible with Sau3A1). Three versions of the pKK LamB-P vector were used in which the lamB polynucleotide is found in each of the three reading frames relative to the multiple cloning site. E. coli host XLOLR—a lambda resistant, supO host strain (Stratagene, Calif., USA) was transformed with the ligated DNA libraries. Stratagene XL1 Blue Manual.

The transfected cells were then infected with λ:: 1105 which is a kanamycin resistant suicide lambda virus. (See Kleckner et al., 1991, Methods Enzymol. 204:139-180.) Lambda virus can only infect cells that have a lambda receptor protein on the surface, i.e., the lamB protein. Since the lamB gene in the pKKLamB cloning vector lacks a secretory leader to direct the receptor to the surface, only those lamB fusions that contain a signal peptide are infectible. The suicide lambda phage, once inside the E. coli cell, can transpose the kanamycin resistance gene randomly to the E. coli chromosome. Thus, a colony can only aquire kanamycin resistance provided the screened polynucleotide encoded a cleavable N-terminal signal sequence.

The following results were obtained when screening an E. coli genomic library. Eight genes were sequenced and corresponded to known E. coli genes (seven were known periplasmic or outer membrane receptors, the remaining one is uncharacterized). Five genes were sequenced and corresponded to putative E. coli transport or receptor proteins based on homology to other E. coli proteins. Fifteen genes were in the E. coli database but no known function has been ascribed yet.

None of the identified clones was of a type that should not have been identified with the methods of the invention, i.e., internal methionines, N-termini from non-secreted gene products, or noncoding DNA segments. Thus, the described methods are effective in identifying desired polynucleotide clones and in excluding undesired polynucleotide clones.

4. Screening a Salmonella typhi Genomic Library

A genomic library from pathogenic Salmonella typhi was prepared and screened as described in Example 5(G)3. Seventeen clones contained DNA that was not in any database. Seven clones yielded DNA that could be identified as either surface proteins of Salmonella or as homologs of E. coli proteins that were located on the surface.

### 5(H) Example

### Identification of Bacterial Type III Secreted Proteins

1. Background

The pathogenic response elicited by many bacteria when infecting mammalian cells involves the invasion of the host cells by cytotoxic proteins (i.e., invasins) encoded by the bacteria (for a review, see, Cornelius, 1998, J. Biotechnol. 180:5495-5504). Developing a rapid method to discover the genes encoding the invasins is useful for the development of therapeutics or antiseptics to neutralize these toxins and diagnostics for their detection. Invasins are secreted by pathogenic bacteria following a signal generated by their close proximity to the mammalian cell (Cornelius, 1998, supra). The signal is transmitted via a membrane receptor that is present on the surface of the bacteria. These receptor molecules (containing N-terminal signal peptides) may be identified with the methods of the invention.

However, this method for cloning secreted proteins typically cannot be directly applied to the invasins because they are secreted by a different mechanism that does not utilize a traditional N-terminal signal peptide that is cleaved. These proteins are secreted by the Type III secretory apparatus which involves a number of host-encoded proteins and does not result in the N-terminal cleavage of the protein that is exported (Hueck, 1998, supra). Although these molecules are not N-terminally processed, it has been demonstrated that either the 5′ end of the mRNA or the N-terminus of the protein carries a determinant(s) to permit their secretion (Anderson et al., 1997, Science 278:1140-1143). These segments do not contain a consensus sequence, therefore, they cannot be readily identified by sequence analysis. Furthermore, it has been shown that if the N-terminal segment of a Type III secreted protein is fused to a second polypeptide that is not secreted in this manner, the N-terminus can direct the hybrid protein to be secreted by the Type III pathway (Michiels et al., 1991, J. Bacteriol. 173:1677-1685).

Since Type III secretion typically requires the presence of many host proteins in the pathogenic bacteria, transposing this entire secretory apparatus directly into E. coli may pose a significant hurdle. With one approach, one would establish the secretory apparatus in E. Coli for each organism of interest prior to screening. The approach of the present invention converts the lamB selection into one that can function directly in gram-negative bacteria that express the set of host proteins involved in Type III secretion.

The bacteriophage lambda can infect E. coli by virtue of the LamB receptor present on its surface. Once lambda is inside the E. coli, many E. coli proteins are involved in its propagation. However, entry merely requires presence of LamB on the surface. The inventors have determined that lambda can infect many cell types (including mammalian CHO cells) if the LamB receptor is expressed on the cell surface. See U.S. patent application Ser. No. 08/834,134, filed Apr. 14, 1997. Thus, it is likely that the LamB receptor, when expressed on the cell surface of a pathogenic gram-negative bacterium, renders the cell susceptible to lambda phage infection.

2. Screening for Type III Secreted Bacterial Proteins

Plasmids of a class called broad host range are capable of conjugal mating and stable propagation in all species of gram negative bacteria (Thomas et al., 1987, Ann. Rev. Micro. 41:77-101). RK2 is a broad host range plasmid of 60 kb in size (Thomas et al., 1987, supra). The minimal requirements for replication of RK2 consist of an origin of replication (oriV, a 393 bp segment comprised if direct repeats) and a replication protein called TrfA (Thomas et al., 1987, supra). In addition, conjugal mating of this plasmid into other gram-negative bacteria typically requires that the plasmid contain an origin of conjugal transfer (oriT), a 140 bp segment comprised of binding/nicking sites for a number of mating proteins (Guiney et al., 1988, Plasmid 20:259-265). The mating proteins of RK2 (approximately 25 gene products encompassing 30 kb) are encoded on the plasmid to ensure self-transmissibility. However, it has been demonstrated that these transfer proteins can be present in trans (on the host chromosome or a second plasmid) and can efficiently mobilize an oriT containing plasmid that lacks the transfer proteins (Ditta et al., 1980, Proc. Natl. Acad. Sci. USA 77:7347-7351).

First, it is determined whether the LamB receptor, if expressed in these pathogenic gram-negative bacterial hosts, permits the lambda virus to infect. For this analysis, one uses the pKK LamB-P plasmid expressing the LamB with an N-terminal signal peptide. For this purpose, a positive clone identified in Example 5(G)2 is used. This plasmid is transformed into CAG1000, a prototrophic E. coli strain that is recA⁺ (Singer, 1989, Microbiol. Rev. 53:3-53). This strain is then transformed with pAL37 DNA (Amp^{R}, Kan^{R}), a derivative of RK2 (compatible with the colE1 origin of pKK LamB) that is deleted for its native tet^{R} gene (Greener et al., 1992, Genetics 130:27-36). Selection for kanamycin resistance is carried out.

Because the two plasmids (pAL37 and pKK LamB-P) share homology with one another (the ampR gene) and since the host is Rec⁺, a certain percentage of the plasmids recombine with one another forming a plasmid cointegrate (estimated to be approximately 1%). A population of the CAG cells carrying both plasmids is then mated with XLOLR (which is nalidixic acid resistant) and exconjugants are selected on plates with nalidixic acid and tetracycline. The pAL37 DNA can readily mobilize itself into XLOLR. The pKK LamB-P plasmid, which lacks the origin of transfer, cannot be conveyed into the recipient unless it became a “passenger” on the RK2 via cointegrate formation. The pool of exconjugates contains predominantly the RK2 plasmid only. However, a small percentage should harbor the cointegrate plasmid. These cointegrates can be readily retrieved by infecting the XLOLR pool with λgt10 Tet^{R} (identical to λgt10 Kan^{R} except for drug resistance gene) and selecting for Tet^{R} lysogens since only those XLOLR cells that received a cointegrate plasmid carry a functional lamB gene.

This cointegrate plasmid (stable in the recA⁻ XLOLR strain) can then be mated into the gram negative host strain of choice (i.e., Yersinia, Salmonella, Pseudomonas) and selection for the Amp resistance or Kan resistance (whichever is appropriate) on media that permits only the recipient cell to grow. Media compositions specifically for this purpose have been devised for many gram-negative bacteria—for others, their natural resistance to compounds like rifampicin or streptomycin can also be used (Schmidhauser et al., 1985, J. Bacteriol. 164:446-455). It has been shown that most gram negative bacteria are sensitive to tetracycline (Schmidhauser et al., 1985, supra). Thus, this drug resistance gene was designated for selection of lambda infectivity.

It is likely that the signal peptide that precedes LamB is functional since it was derived from the gram-negative bacterium under investigation. When the fusion is expressed in the pathogenic bacterium, the LamB protein should be translocated to the periplasmic space. In order for the inventive system to be viable, the LamB protein typically must enter the outer membrane and assemble there in a manner similar to its assembly in E. coli. If this were to occur, then the host bacterium would be infectible by lambda. This is assayed by infection with λ1098 carrying the tetR gene as part of a transposable element (Kleckner et al., 1991, Methods Enzymol. 204:139-180). If the host cells become TetR upon infection, it can be concluded that they were infectible by lambda and that the LamB protein had the ability to function in this heterologous environment. If cells are noninfectible, the membrane protein fraction is electrophoresed through a polyacrylamide gel and probed with both anti-lamB antibodies (Stratagene, Calif., USA) and anti-FLAG antibodies (Stratagene, Calif., USA) to determine whether the LamB protein is present at the cell surface. It is possible that the LamB protein is membrane bound but oriented in a manner that renders it non-infectible by lambda.

In order to identify polynucleotides that encode Type III signal sequences, a type III secretory leader from Yersinia pestis (a yop gene) (Cornelius, 1998, J. Bacteriol. 180:5495-5504) is inserted into the original pKK LamB-P cloning vector, and the plasmid cointegrate experiment is repeated. This cointegrate, when present in Yersinia should secrete the LamB protein as a fusion polypeptide (Micheils et al., 1991, J. Bacteriol. 173:1677-1685). Normally, proteins destined for secretion by the Type III apparatus become extracellular. However, because of the membrane spanning motifs that comprise LamB, the fusion protein may become tethered to the outer membrane. Then, depending on the leader sequence (size, amino acid composition, etc.), it may also be possible that the LamB protein may be oriented in a manner that permits lambda to infect. This is tested using λ1098 as above. Generation of Tet^{R} colonies indicates successful infection by lambda. If initially unsuccessful, a second Type III secretory leader from Yersinia is tried. If cells expressing this fusion remain noninfectible by lambda, the cells are probed with antibodies generated against either LamB or FLAG to determine the fate of the LamB fusion polypeptide.

If the LamB fusion polypeptide is retained in the outer membrane, but is not correctly positioned to permit phage infection, it may be possible to retrieve clones expressing the bound LamB using antibodies to LamB. This is carried out as follows. Cells containing the plasmid expressing the LamB fusion are mixed in varying ratios with cells containing the starting LamB plasmid. The mixed population is then treated with LamB antibodies in a standard immunoprecipitation experiment. The recovered cells are then plated for single colonies and the restriction digest pattern of the plasmids in the colonies using at least one restriction enzyme is determined to distinguish the different colonies. Enrichment for the LamB membrane tethered cells is thus evaluated qualitatively and quantitatively.

If the LamB fusions with N-terminal Type III secretory leaders are either lambda infectible or selectable using antibodies, a series of vector alterations is introduced to explore the possibility of converting pKK LamB vector to a plasmid for broad host range library screening. Because the cointegrate strategy described above for a single plasmid may not be efficient enough for an entire library screen, the pKK LamB-P plasmid is modified to enable it to be directly introduced into the gram negative host via conjugal mating. The origin of replication (oriV) (Thomas et al., 1987, Ann. Rev. Micro. 41:77-101), the TrfA replication protein (trfa) (Thomas et al., 1987, supra), and the origin of transfer (oriT) (Guiney et al., 1988, Plasmid 20:259-265) are inserted into the pKK LamB-P vector by conventional restriction enzyme cloning of PCR generated segments. Each inserted element is functionally tested to ensure that no mutation was introduced during the PCR amplification. The conjugal transfer functions are supplied, in trans, by E. coli harboring a helper plasmid (pRK2013) (Ditta et al., 1980, Proc. Natl. Acad. Sci. USA 77:7347-7351).

Libraries prepared using genomic DNA from P. aeruginosa digested with Sau3A or Tsp509I are constructed using the newly modified pKK LamB vector (3 vectors, i.e., one for each reading frame, yielding 6 libraries total). The initial library ligation is transformed into XL10-GOLD that contains the helper plasmid pRK2013. After 60 minutes growth at 37° C. to permit establishment of the plasmid library, the cells are then plated onto agar plates that have been spread with a logarithmic culture of P. aeruginosa. These agar plates contain both ampicillin (to select for the LamB plasmids) and streptomycin to select against the sensitive E. coli. Because mating by the RK2 system is optimal on agar plates rather than in a shaking culture (Ditta et al., 1980, Proc. Natl. Acad. Sci. USA 77:7347-7351), colonies that arise should represent P. aeruginosa cells containing the pKK LamB plasmid library. The colonies are then pooled, grown briefly in selective media, and then they are infected with λ1098 (selection to Tet^{R} at levels that are empirically determined) or immunoprecipitated with LamB antibodies. This should retrieve Pseudomonas cells that have LamB on their surface. Plasmid DNA from positive clones are isolated and the inserts sequenced. If the proposed system were to function as described here, genes that are secreted by both the traditional N-terminal pathway and those that are secreted by the Type III pathway should be present.

The present invention is not to be limited in scope by the exemplified embodiments which are intended as illustrations of single aspects of the invention, and any clones, DNA or amino acid sequences which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims. It is also to be understood that all base pair sizes given for nucleotides are approximate and are used solely for purposes of description.

All documents cited herein are incorporated by reference in their entirety for any purpose. The citation of any of the documents mentioned herein does not constitute an admission that the reference is prior art to the present invention.

What is claimed is:

1. A method of screening for a polynucleotide encoding a cleavable N-terminal signal sequence comprising
culturing a cell containing a screening vector, wherein the vector comprises screened polynucleotide and marker polynucleotide encoding a cell surface protein that will not be associated with the cell surface unless the marker polynucleotide encoding the cell surface protein is fused to screened polynucleotide encoding a cleavable N-terminal signal sequence and the fused polynucleotides are expressed to produce a fusion protein comprising a cleavable N-terminal signal sequence and the cell surface protein; and
exposing the cell to an agent that will confirm whether the cell surface protein is located on the surface of the cell.

2. The method of claim 1 wherein the cell is a prokaryotic cell.

3. The method of claim 2 wherein the marker polynucleotide encodes a cell surface receptor and the agent interacts with the cell surface receptor.

4. The method of claim 3 wherein the cell surface receptor is lamB protein or a lamB protein analog and the agent is a phage or virus that infects cells that have lamB protein or lamB protein analog on the cell surface.

5. The method of claim 4 wherein the phage or virus comprises a marker that confers a detectable property on cells that the phage or virus infects.

6. The method of claim 5 wherein the phage or virus comprises a marker that confers antibiotic resistance on cells that the phage or virus infects.

7. The method of claim 6 wherein the cells that have been exposed to the phage or virus are exposed to the antibiotic to which the phage or virus confers antibiotic resistance.

8. The method of claim 7 wherein the phage or virus comprises a marker that confers resistance to at least one of kanomycin, tetracycline, streptomycin, chloramphenicol, gentamycin, or hygromycin on cells that the phage or virus infects.

9. The method of claim 8 wherein the cell surface receptor is lamB protein and the agent is lambda phage.

10. The method of claim 9 wherein the prokaryotic cell is E. coli.

11. The method of claim 7 wherein polynucleotide encoding the cell surface protein from cells that survive exposure to the antibiotic is sequenced to determine additional nucleotide sequence of polynucleotide that was fused to it.

12. The method of claim 3 wherein the cell surface receptor is a receptor that allows uptake into the cell of a given nutrient and the agent is the given nutrient.

13. The method of claim 12 wherein the cells are cultured on a medium that requires cells to uptake the given nutrient from the media in order to survive.

14. The method of claim 13 wherein the given nutrient is at least one of maltose, Vitamin B₁₂, or iron.

15. The method of claim 13 wherein polynucleotide encoding the cell surface protein from cells that survive culturing on the medium comprising the given nutrient is sequenced to determine additional nucleotide sequence of polynucleotide that was fused to it.

16. The method of claim 2 wherein the agent is a detectable ligand that interacts only with cells that include the cell surface protein on the surface of the cell.

17. The method of claim 16 wherein the detectable ligand is a labeled antibody specific for the cell surface protein.

18. The method of claim 17 wherein polynucleotide encoding the cell surface protein from cells detected with the labeled antibody is sequenced to determine additional nucleotide sequence of polynucleotide that was fused to it.

19. A method of screening for a polynucleotide encoding a cleavable N-terminal signal sequence comprising
exposing a library of polynucleotides to a screening vector, wherein the screening vector comprises marker polynucleotide, wherein the marker polynucleotide is capable of being fused to screened polynucleotides upon exposure to them and wherein the marker polynucleotide encodes a cell surface protein that will not be associated with a cell surface unless the marker polynucleotide encoding the cell surface protein is fused to screened polynucleotide encoding a cleavable N-terminal signal sequence and the fused polynucleotides are expressed to produce a fusion protein comprising a cleavable N-terminal signal sequence and the cell surface protein;
transferring the screening vector that has been exposed to the library of polynucleotides into a cell;
culturing the cell; and
exposing the cell to an agent that will confirm whether the cell surface protein is located on the surface of the cell.
